(19) United States
(12) Patent Application Publication
(10) Pub. No.: US 2022/0401101 A1
(43) Pub. Date: Dec. 22, 2022

(54) PURSE-STRING APPLICATOR FOR CIRCULAR STAPLING INSTRUMENTS

(71) Applicant: Covidien LP, Mansfield, MA (US)

(72) Inventor: Justin Williams, Southbury, CT (US)

(21) Appl. No.: 17/349,390

(22) Filed: Jun. 16, 2021

(51) Int. Cl. A61B 17/115; A61B 17/11

(52) U.S. Cl. CPC A61B 17/1155; A61B 17/1114; A61B 2017/00424

(57) ABSTRACT

A purse-string applicator for use in an anastomosis procedure includes an introducer sleeve, a handle assembly, and a support ring. The introducer sleeve includes a tubular body portion having proximal and distal portions. A handle portion is disposed on the proximal portion of the tubular body, and an introducer portion is disposed on the distal portion of the tubular body. The handle assembly includes an elongate shaft portion and a release mechanism. The release mechanism is positionable distal of the distal portion of the introducer sleeve. The support ring is releasably engageable by the release mechanism of the handle assembly and includes an annular body portion and first and second rim portions. The release mechanism releasably retains the support ring adjacent the distal portion of the introducer sleeve. The purse-string applicator may be included in a kit along with an anvil assembly, an adapter assembly, and/or a circular stapling instrument.

### FIELD

The disclosure relates to circular stapling instruments for creating anastomoses. More particularly, the disclosure relates to an applicator for facilitating formation of an anastomosis using a double purse-string suture technique.

### BACKGROUND

Circular stapling instruments are used to perform end to end anastomosis procedures. During a typical end-to-end anastomosis procedure, a loading unit of the circular stapling instrument is secured to a first section of a tubular organ and an anvil assembly of the circular stapling instrument is secured to a second section of the tubular organ. A purse-string suture may be used to secure the first and second sections of the tubular organ to the loading unit and the anvil assembly of the circular stapling instrument, respectively. A second purse-string suture may also be used to secure the second section of the tubular organ to the loading unit. This double purse-string suture technique may be preferable to other known tubular organ securing techniques because, unlike traditional tubular organ securing techniques, there is no staple line in the tubular organ to cut through. In addition, the double purse-string suture technique prevents or inhibits the creation of undesirable “dog ear” pockets at the corner of the linear staple line created with the traditional securing techniques.

Using known double purse-string securing techniques, a purse-string suture is applied to the second section of the tubular organ to secure the second section of the tubular organ about the loading unit which is on the distal end of the circular stapling instrument. Once the second section of tubular organ is secured to about the loading unit, the circular stapling instrument must remain in position until fired. The presence of the circular stapling instrument within the tubular organ obstructs the field of vision of a clinician. Further, maintaining the circular stapling instrument within the tubular organ of the patient for any length of time increases trauma to the patient.

It would be beneficial to have an apparatus for forming a purse-string suture in a section of a tubular organ independent of the circular stapling instrument.

### SUMMARY

A purse-string applicator for use in an anastomosis procedure includes an introducer sleeve, a handle assembly, and a support ring. The introducer sleeve includes a tubular body portion having proximal and distal portions. A handle portion is disposed on the proximal portion of the tubular body, and an introducer portion is disposed on the distal portion of the tubular body. The handle assembly includes an elongate shaft portion and a release mechanism. The release mechanism is positionable distal of the distal portion of the introducer sleeve. The support ring is releasably engageable by the release mechanism of the handle assembly and includes an annular body portion and first and second rim portions. The release mechanism releasably retains the support ring adjacent the distal portion of the introducer sleeve.

In certain aspects of the disclosure, the introducer portion of the introducer sleeve includes a bulbous portion that is configured to facilitate entry of the bulbous portion into a body cavity of a patient. The introducer portion of the introducer sleeve may define a recess. The support ring may be received within the recess. The handle portion of the introducer sleeve may be configured to facilitate grasping by a clinician.

In other aspects of the disclosure, the elongate shaft portion of the handle assembly defines a pair of cutouts and the release mechanism may include a pair of ball-bearings received within the pair of cutouts. The release mechanism may include a pair of springs and the pair of ball-bearings may be biased outwardly by the pair of springs into engagement with the support ring. The release mechanism may include a hub and a plurality of flexible legs extending from the hub. The plurality of flexible legs may be transitionable between a bulbous condition and a substantially cylindrical condition. The plurality of flexible legs may be configured to releasably engage the support ring when in the bulbous condition. Retraction of the handle assembly relative to the introducer sleeve may transition the plurality of flexible legs from the bulbous condition to the substantially cylindrical condition.

A kit for use in an anastomosis procedure includes an anvil assembly and a purse-string applicator. The purse-string applicator includes an introducer sleeve, a handle assembly, and a support ring. The introducer sleeve includes a tubular body portion having proximal and distal portions. A handle portion is disposed on the proximal portion of the tubular body, and an introducer portion is disposed on the distal portion of the tubular body. The handle assembly includes an elongate shaft portion and a release mechanism. The release mechanism is positionable distal of the distal portion of the introducer sleeve. The support ring is releasably engageable by the release mechanism of the handle assembly and includes an annular body portion and first and second rim portions. The release mechanism releasably retains the support ring adjacent the distal portion of the introducer sleeve.

A kit for use in an anastomosis procedure includes a circular stapling instrument, an anvil assembly releasably securable to the circular stapling instrument, and a purse-string applicator.

A method of performing an anastomosis using a double purse-string suture technique includes, positioning a support ring into a tubular organ of a patient with a purse-string applicator, securing a first section of the tubular organ to the support ring with a first purse-string suture, separating the support ring from the purse-string applicator, removing the purse-string applicator from the tubular organ, positioning a distal portion of a circular stapling instrument, including a loading unit and trocar member, into the first section of the tubular organ such that the trocar member extends through the support ring, positioning the support ring and attached first section of the tubular organ within the loading unit of the circular stapling instrument, and actuating the circular stapling instrument.

In certain aspects of the disclosure, securing the first section of the tubular organ to the support ring includes forming the purse-string suture about an annular body portion of the support ring. Separating the support ring from the purse-string applicator may include retracting the handle assembly of the purse-string applicator relative to an introducer sleeve of the purse-string applicator. Removing the purse-string applicator from the tubular organ may include retracting the introducer sleeve. Positioning the distal portion of the circular stapling instrument into the first section of the tubular organ may include introducing proximal portion of the purse-string applicator through an anus of a patient.

In other aspects of the disclosure, the method further includes securing the support ring to the purse-string applicator. Actuating the circular stapling instrument may include stapling and cutting the tubular organ. Actuating the circular stapling instrument may include forming an anastomosis donut.

### BRIEF DESCRIPTION OF THE DRAWINGS

The accompanying drawings, which are incorporated in and constitute a part of this specification, illustrate aspects of the disclosure and, together with a general description of the disclosure given above, and the detailed description given below, explain the aspects of the disclosure, wherein:

FIG. 1 is a perspective side view of a circular stapling instrument and a purse-string applicator according to aspects of the disclosure;

FIG. 2 is a perspective side view of the purse-string applicator shown in FIG. 1;

FIG. 3 is a perspective side view of the purse-string applicator shown in FIGS. 1 and 2, with components separated;

FIG. 4 is an enlarged view of the indicated area of detail shown in FIG. 2;

FIG. 5 is a perspective side view of a support ring of the purse-string applicator shown in FIGS. 1 and 2;

FIG. 6 is a cross-sectional view taken along section line 6-6 of the purse-string applicator shown in FIG. 2;

FIG. 7 is an enlarged view of the indicated area of detail shown in FIG. 6;

FIG. 8 is a side view of a distal portion of the purse-string applicator (shown in phantom) introduced into a first section of a tubular organ;

FIG. 9 is a side view of the distal portion of the purse-string applicator shown in FIG. 8 positioned within the first section of the tubular organ with a purse-string suture securing the tubular organ to the distal portion of the purse-string applicator and the purse-string applicator shown in phantom;

FIG. 10 is a cross-sectional side view of the first section of the tubular organ secured to the support ring of the purse-string applicator as a handle assembly of the purse-string applicator is removed from within an introducer sleeve of the purse-string applicator;

FIG. 11 is a cross-sectional side view shown in FIG. 10 with the introducer sleeve of the purse-string applicator removed and the first section of the tubular organ secured to the support ring by the purse-string suture;

FIG. 12 is a side view of the first section of the tubular organ and the support ring shown in FIG. 11 with a distal portion of the circular stapling instrument shown in FIG. 1 positioned within the first section of the tubular organ and extending though the support ring;

FIG. 13 is a cross-sectional side view of the tubular organ, the support ring, and the distal portion of the circular stapling instrument with a trocar member of the circular stapling instrument extending through the support ring;

FIG. 14 is the cross-sectional side view of the tubular organ, the support ring, and the distal portion of the circular stapling instrument, further including an anvil assembly secured to the distal portion of the circular stapling instrument in a clamped position with the anvil assembly positioned with a second section of the tubular organ (shown in phantom);

FIG. 15 is a perspective end view of a purse-string applicator according to other aspects of the disclosure;

FIG. 16 is a cross-sectional side view taken along section line 16-16 of the purse-string applicator shown in FIG. 15; and

FIG. 17 is the cross-sectional view of the purse-string applicator shown in FIG. 16, as a handle assembly of the purse-string applicator is retracted within an introducer sleeve of the purse-string applicator.

### DETAILED DESCRIPTION

Aspects of the disclosure are described in detail with reference to the drawings, in which like reference numerals designate identical or corresponding elements in each of the several views. As used herein, the term “distal” refers to that portion of the surgical instrument, or component thereof, that is farther from the user during use of the instrument in its customary fashion, while the term “proximal” refers to that portion of the surgical instrument, or component thereof, that is closer to the user during use of the instrument in its customary fashion. As used herein, the term clinician refers to anyone involved in a surgical procedure, including but not limited to, surgeons, support staff, and other medical personnel. As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

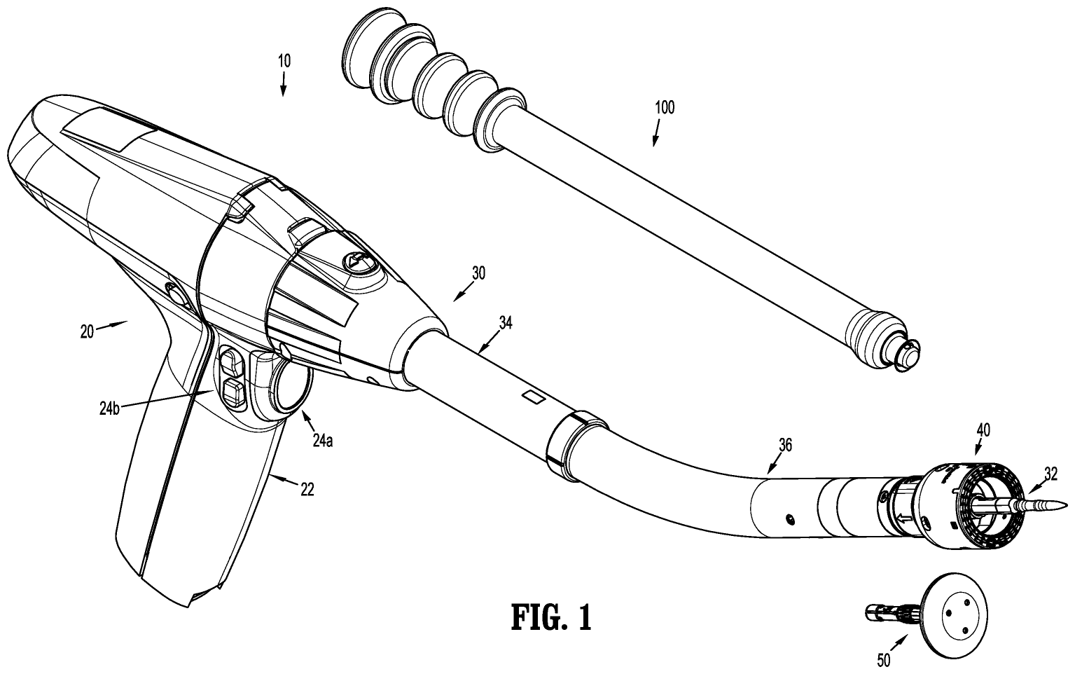

FIG. 1 illustrates a circular stapling instrument 10, and a purse-string applicator according to aspects of the disclosure, shown generally as purse-string applicator 100. The circular stapling instrument 10 includes a powered handle assembly 20, an adapter assembly 30 that is releasably secured to the powered handle assembly 20, a loading unit 40 that is secured to a distal portion 36 of the adapter assembly 20, and an anvil assembly 50 that is releasably securable to a trocar assembly 32 of the adapter assembly 30. Although shown and described with reference to the circular stapling instrument 10, the aspects of the disclosure may be modified for use with circular stapling instruments having alternative configurations. For example, the adapter assembly 20 may be configured as a component in a robotic system, or the handle assembly may be configured for manual actuation. The handle assembly 20 includes a stationary grip 22 that supports actuation buttons 24a, 24b for controlling operation of various functions of the circular stapling instrument 10 including, for example, clamping, stapling, and cutting of tissue.

The adapter assembly 30 includes a proximal portion 34 that is configured for operable connection to the handle assembly 20 and a distal portion 36 that is configured for operable connection to the loading unit 40. Although shown as forming an integral unit, it is envisioned that the proximal and distal portions 34, 36 of the adapter assembly 30 may be formed as separate units that are releasably securable to one another.

The circular stapling instrument 10 will only be described to the extent necessary to fully disclose the aspects of the disclosure. For a detailed description of exemplary circular stapling instruments, please refer to U.S. Pat. Nos. 10,226,254 and 10,111,684 (“the '684 patent”).

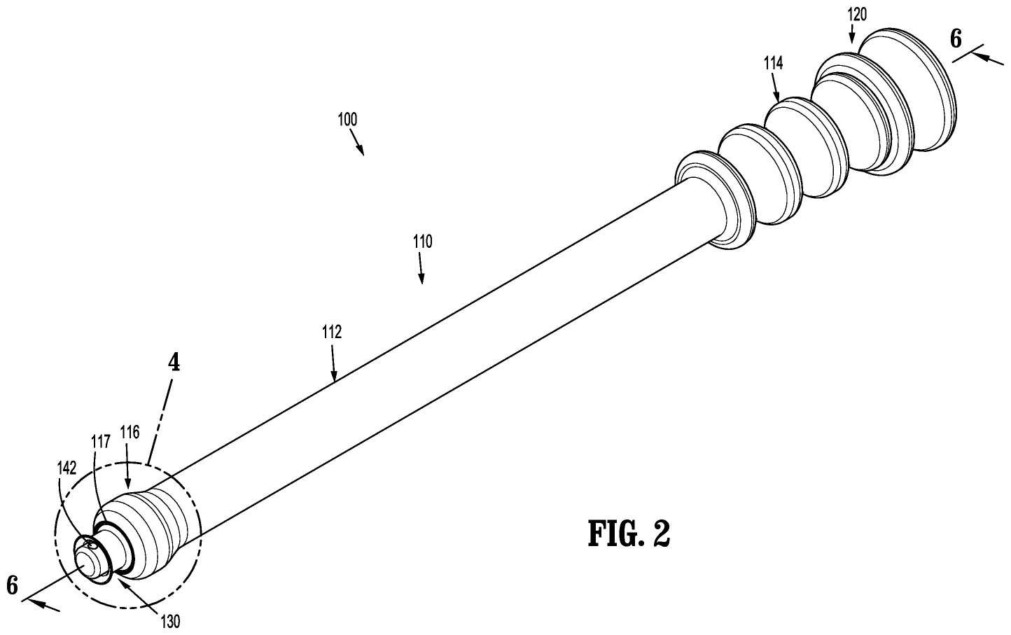

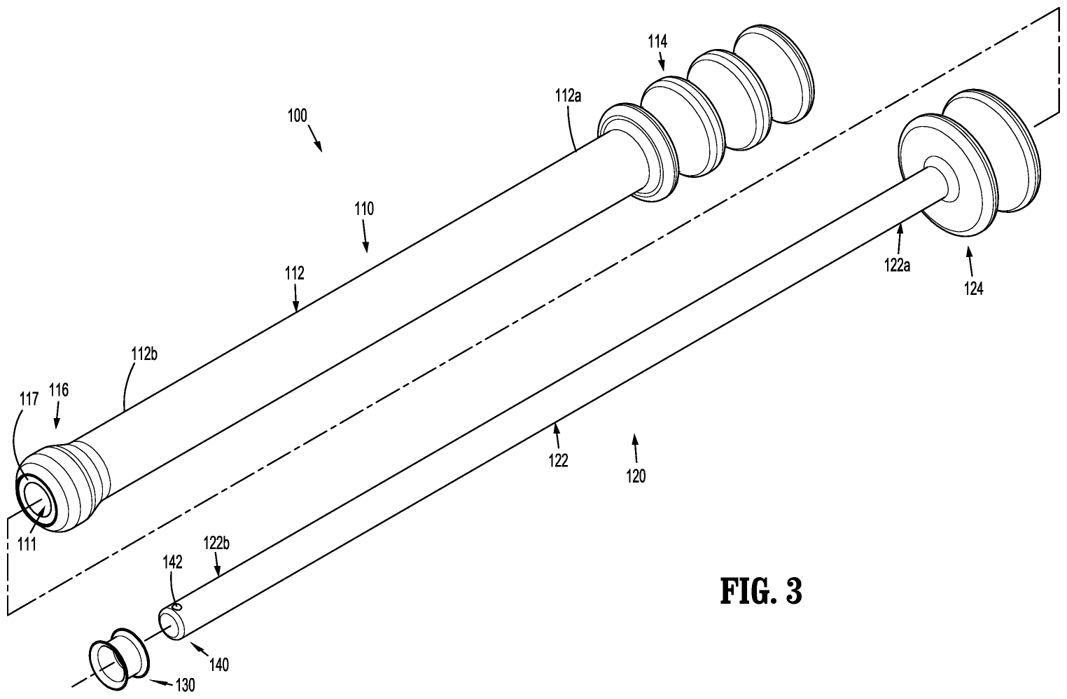

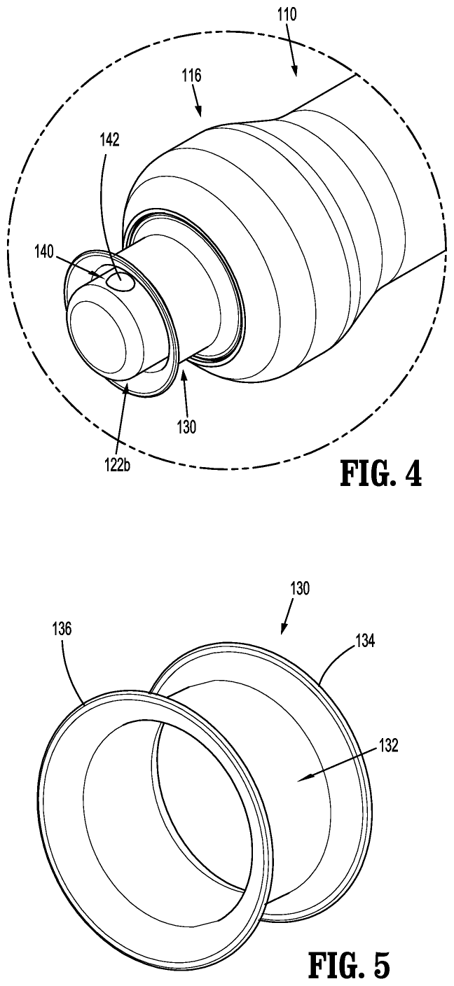

FIGS. 2 and 3 illustrate the purse-string applicator 100 that includes an introducer sleeve 110, a handle assembly 120 that is moveably positionable relative to the introducer sleeve 110, and a support ring 130 that is releasably supported on a distal end of the handle assembly 120 (FIG. 3). It is envisioned that the size and configuration of the introducer sleeve 110, the handle assembly 120, and the support ring 130 may be selected to suit a particular patient, procedure, and/or preference of the clinician.

The introducer sleeve 110 of the purse-string applicator 100 includes a tubular body portion 112, a handle portion 114 that is disposed on a proximal portion 112a of the tubular body portion 112, and a bulbous introducer portion 116 that is disposed on a distal portion 112b of the tubular body portion 112. The introducer sleeve 110 defines a longitudinal passage 111 (FIG. 3) for receiving the handle assembly 130. The handle portion 114 of the introducer sleeve 110 may include knurling, ribs, detents, or other configurations that facilitate grasping of the introducer sleeve 110 by a clinician. The bulbous introducer portion 116 of the introducer sleeve 110 is configured to facilitate introduction of the support ring 130 into a body cavity, e.g., through an anus of a patient. In aspects of the disclosure, the bulbous introducer portion 116 defines an annular recess 117 for receiving a portion of the support ring 130. Although shown as being integrally formed, it is envisioned that the tubular body portion 112, the handle portion 114, and/or the bulbous introducer portion 116 of the introducer sleeve 110 may be individually formed components secured together in any suitable fashion.

The handle assembly 120 of the purse-string applicator 100 includes an elongate shaft portion 122 and a handle portion 124 disposed on a proximal portion 122a (FIG. 3) of the elongate shaft portion 122. A distal portion 122b of the elongate shaft portion 122 supports a release mechanism 140. More particularly, the distal portion 122b of the elongate shaft portion 122 defines a pair of opposed cutouts 141 (FIG. 7). The opposed cutouts 141 are each configured to retain a ball-bearing 142. A spring 144 (FIG. 7) biases each of the ball-bearings 142 radially outward such that a portion of each of the ball-bearings 142 protrudes from one of the opposed cutouts 141. The elongate shaft portion 122 of the handle assembly 120 has a length sufficient to expose the distal portion 122b of the elongate shaft portion 122 from the bulbous introducer portion 116 of the introducer sleeve 110 when the handle assembly 120 is received within the longitudinal passage 111 (FIG. 3) of the introducer sleeve 110.

The support ring 130 (FIG. 5) of the purse-string applicator 100 includes an annular body portion 132 and first and second rim portions 134, 136 that extend from opposite ends of the annular body portion 132. The annular body portion 132 is sized to be received about the distal portion 122b of the elongate shaft portion 122 of the handle assembly 120. As shown, and to facilitate manufacturing and assembly of the purse-string applicator 100, the first and second rim portions 134, 136 of the support ring 130 are symmetric, e.g., the first and second rim portions 134, 136 are the same size and configuration. Alternatively, each of the first and second rim portions 134, 136 of the support ring 130 may be different sizes and/or configurations. The first rim portion 134 of the support ring 130 is sized to be received within the recess 117 (FIG. 3) of the bulbous introducer portion 116 of the introducer sleeve 110. The second rim portion 134 of the support ring 130 is positioned to be engaged by the ball-bearings 142 of the release mechanism 140 of the handle assembly 120 when the first rim portion 134 of the support ring 130 is positioned in the recess 117 of the bulbous introducer portion 116 of the introducer sleeve 110. More specifically, the ball-bearings 142 engage a distal side of the second rim portion 136 of the support ring 130 to retain the support ring 130 on the distal portion 122b of the elongate shaft portion 122 of the handle assembly 120 (FIG. 7). As will be described in further detail below, the annular body portion 132 of the support ring 130 provides a location for tying of a first purse-string suture “PSS1” (FIG. 9) during an anastomosis procedure utilizing the double purse-string suture technique.

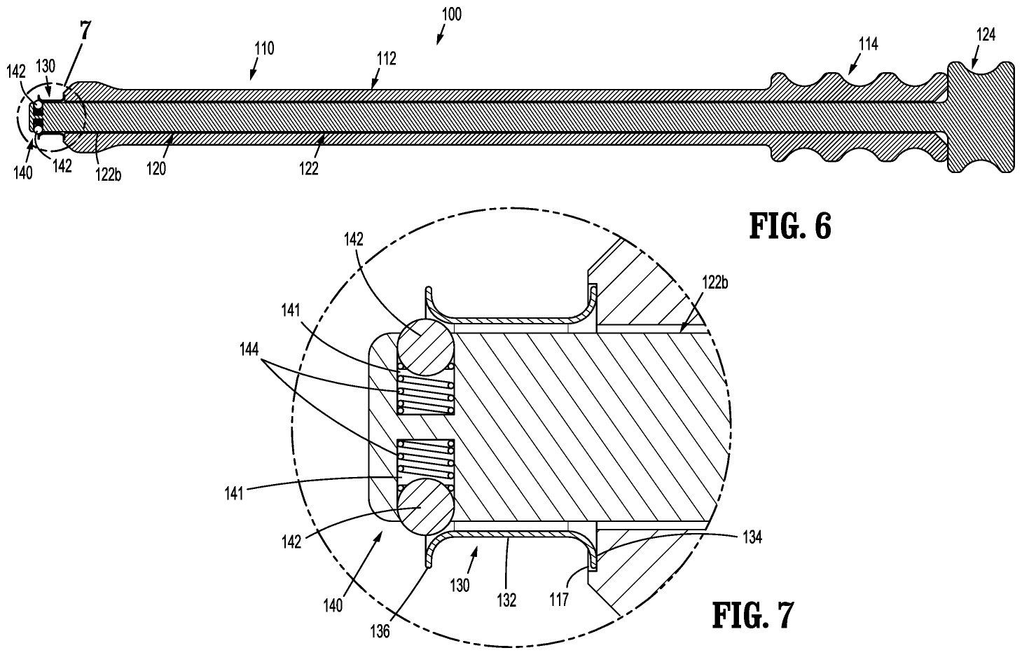

FIGS. 6 and 7 illustrate the purse-string applicator 100 with the handle assembly 120 received with the introducer sleeve 110, and the support ring 130 releasably supported on the distal portion 122b of the elongate shaft portion 122 of the handle assembly 120. More specifically, the first rim portion 134 of the support ring 130 is received within the recess 117 in the bulbous introducer portion 116 of the introducer sleeve 110 and the distal portion 122b of the elongate shaft portion 122 of the handle assembly 120 is received through the support ring 130 such that the ball-bearings 142 of the release mechanism 140 of the handle assembly 120 engage the second rim portion 136 of the support ring 130 to retain the support ring 130 on the distal portion 122b of the elongate shaft portion 122 of the handle assembly 120. The outward bias provided to the ball-bearings 142 by the springs 144 cause the ball-bearings 142 to engage the support ring 130 to releasably secure the support ring 130 relative to the bulbous introducer portion 116 of the introducer sleeve 110.

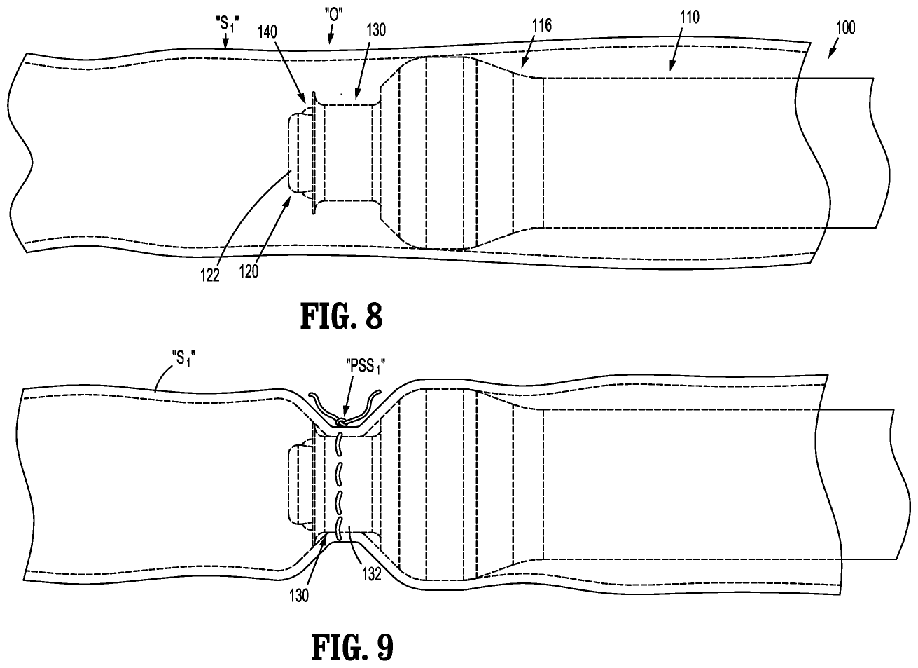

A method of performing an anastomosis procedure with the purse-string applicator 100 utilizing a double purse-string technique will now be described with reference to FIGS. 8-14. It is envisioned that the purse-string applicator 100 may be provided individually or as a kit with the circular stapling instrument 10 (FIG. 1) and/or the anvil assembly 50 (FIG. 1). Support rings 130 having annular body portions 132 of various lengths and diameters may be provided with the purse-string applicator 100. A support ring 130 may be provided to the clinician already secured to the purse-string applicator 100 or the support ring 130 may be secured to the purse-string applicator 100 by the clinician prior to placement of the support ring 130.

FIG. 8 illustrates introduction of a distal end of the purse-string applicator 100 into a first section “S1” of a tubular organ “O”. If not preassembled, the support ring 130 is positioned on the bulbous introducer portion 116 of the introducer sleeve 110 by pushing the support ring 130 in a proximal direction over the ball-bearings 142 of the release mechanism 140 to urge the ball-bearings 142 inwardly against the bias of the springs 144 (FIG. 7). When the second rim portion 136 of the support ring 130 passes proximally of the ball-bearings 142 of the release mechanism 140, the ball-bearings 142 of the release mechanism 140 spring outwardly into engagement with the second rim portion 136 of the support ring 130 to secure the support ring 130 on the distal portion 122b of the elongate shaft portion 122 such that the first rim portion 134 is received in the recess 117 of the introducer sleeve 110. The bulbous configuration of the bulbous introducer portion 116 of the introducer sleeve 110 facilitates atraumatic introduction of the purse-string applicator 100 into the tubular organ “O”.

FIG. 9 illustrates a first purse-string suture “PSS1” sutured into the first section “S1” of the tubular organ “O” about the annular body portion 132 of the support ring 130. The first purse-string suture “PSS1” may include any suitable configuration, e.g., a double pass. Once the purse-string suture “PSS1” is applied to the first section “S1” of the tubular organ “O”, ends of the purse-string suture “PSS1” are tied together.

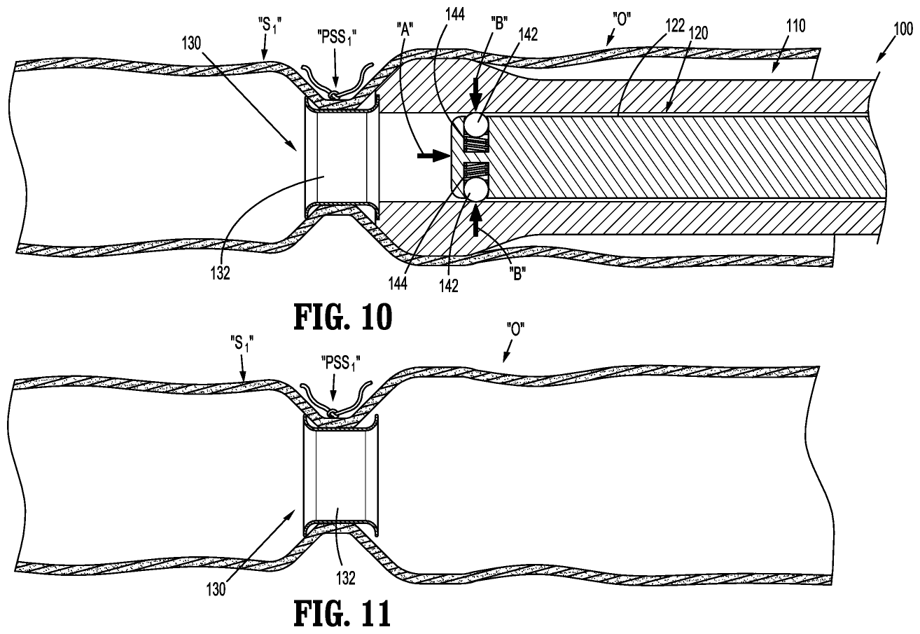

FIG. 10 illustrates release of the support ring 130 of the purse-string applicator 100 from the handle assembly 120 of the purse-string applicator 100. More particularly, retraction of the handle assembly 120 relative the introducer sleeve 110, as indicated by arrow “A” in FIG. 10, causes the annular body portion 132 of the support ring 130 to push against the ball-bearings 142. When this occurs, the springs 144 of the release mechanism 140 compress as the ball-bearings 142 are pressed radially inward by the annular body portion 132 of the support ring 130, as indicated by arrows “B” such that the ball-bearings 142 of the release mechanism 140 pass through the support ring 130.

Although the support ring 130 of the purse-string applicator 100 is shown and described as being secured to the handle assembly 120 through unaided operation of the ball-bearings 142 and the springs 144, it is envisioned that the handle assembly 120 may include a remote actuated release mechanism for causing the release of the support ring 130 from the handle assembly 120.

FIG. 11 illustrates the first section “S1” of the tubular organ “O” after the introducer sleeve 110 of the purse-string applicator 100 is removed from the tubular organ “O”, leaving the support ring 130 secured within the first section “S1” of the tubular organ “O” via compression of organ “O” onto the support ring 130 by the first purse-string suture “PSS1”. Unlike in a traditional double purse-string procedure, the circular stapling instrument 10 does not need to be positioned within the tubular organ “O” in order to form the first purse-string suture “PSS1”. In this manner, the clinician is able to apply the purse-string suture “PSS1” to the first section “S1” of the tubular organ “O” within the patient unobstructed by the circular stapling instrument 10 and without risk of accidentally contacting the circular stapling instrument 10.

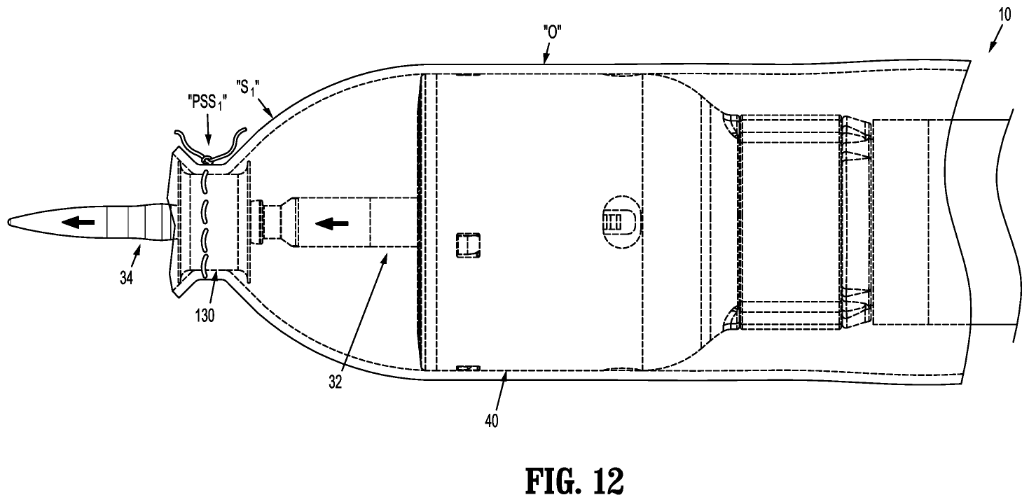

FIG. 12 illustrates a distal portion of the circular stapling instrument 10 (FIG. 1) received through the first section “S1” of the tubular organ “O”. More specifically, the loading unit 40 of the circular stapling instrument 10 is positioned within the first section “S1” of the tubular organ “O” such that a trocar member 34 of the trocar assembly 32 is received through support ring 130. Prior, or subsequent, to receiving the trocar member 34 through the support ring 130, the first section “S1” of the tubular organ “O” may be trimmed to remove excess tissue that might interfere with operation of the circular stapling instrument 10.

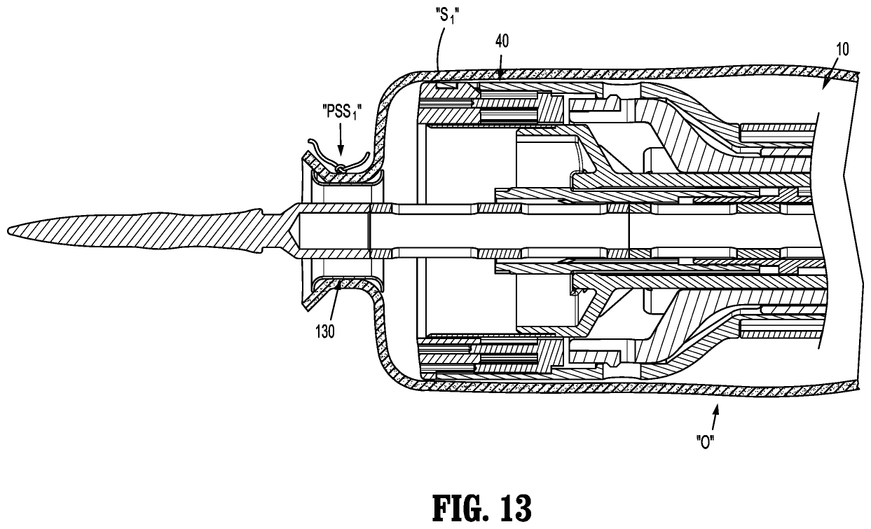

FIG. 13 illustrates the distal portion of the circular stapling instrument 10 (FIG. 1) with the support ring 130 of the purse-string applicator 100, and the first section “S1” of the tubular organ “O” with the first purse-string suture “PSS1”, received about the trocar member 34 of the trocar assembly 32. The support ring 130 may be manually positioned within the loading unit 40 by the clinician. Alternatively, the support ring 130 is positioned within the loading unit 40 through operation of the circular stapling instrument 10, i.e., as the anvil assembly 50 is retracted relative to the loading unit 40, the anvil assembly 50 engages the support ring 130 and pushes the support ring 130 into the loading unit 40.

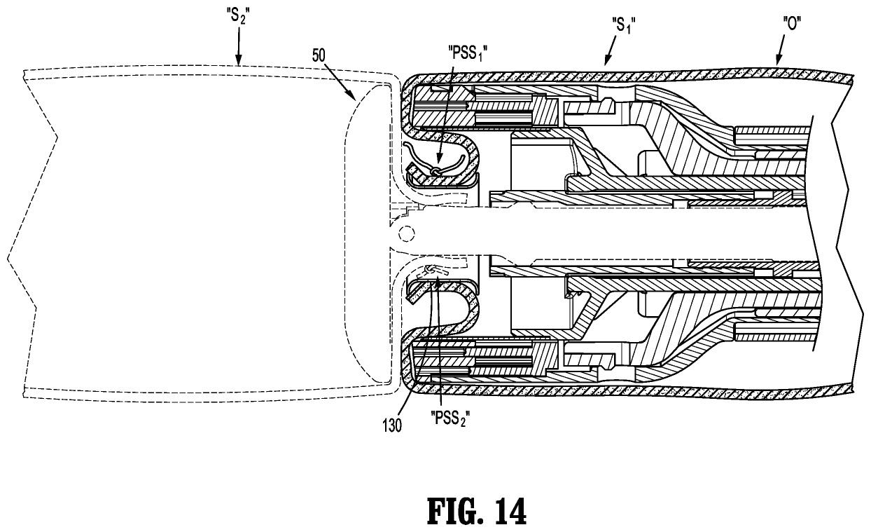

FIG. 14 illustrates the distal portion of the circular stapling instrument 10, including a second section “S2” (shown in phantom) of the tubular organ “O” secured to the anvil assembly 50 (shown in phantom) with a second purse-string suture “PSS2” (shown in phantom), in a clamped condition, and prior to stapling and cutting.

The anastomosis procedure utilizing the double purse-string suture technique is completed in a traditional manner, with the stapling and cutting of tissue occurring simultaneously or sequentially. Once the anvil assembly 50 is separated from the circular stapling instrument 10, the support ring 130 is removed along with an anastomosis donut, i.e., the tissue remaining in the loading unit 40 after the stapling and cutting of the first and second section “S1”, “S2”, as is customary in anastomosis procedures.

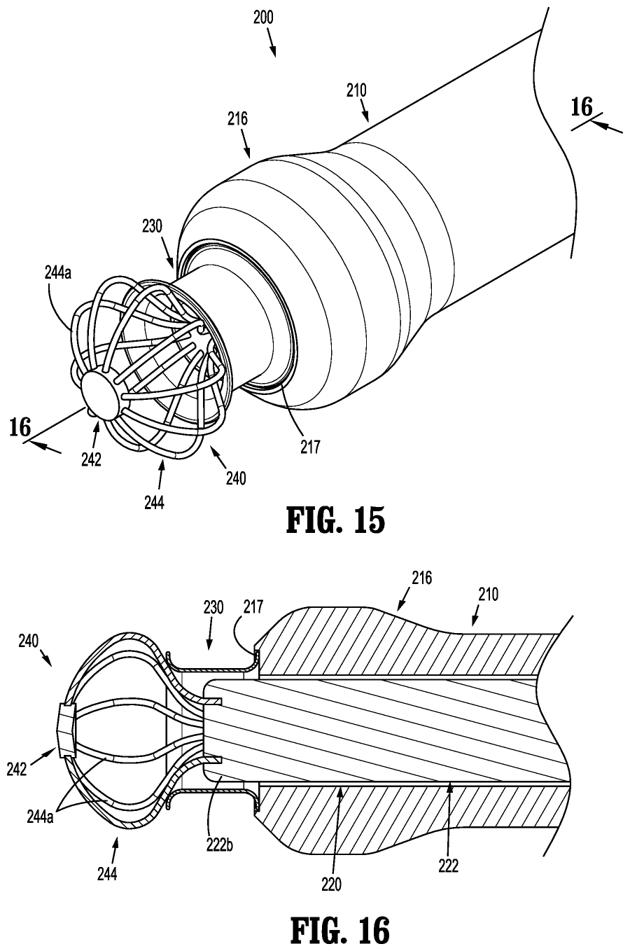

FIGS. 15-17 illustrate a purse-string applicator according to other aspects of the disclosure, shown generally as purse-string applicator 200. The purse-string applicator 200 is substantially similar to the purse-string applicator 100 described hereinabove and will only be described in detail as relates to the differences therebetween.

The purse-string applicator 200 includes an introducer sleeve 210, a handle assembly 220, and a support ring 230. The introducer sleeve 210 and the support ring 230 are substantially similar to the introducer sleeve 110 and the support ring 130 described above and will not be described in further detail herein. Similarly, the proximal portion (not shown) of the handle assembly 220 is substantially similar to the proximal portion of the handle assembly 120. The distal portion of the handle assembly 220 includes a release mechanism 240.

The release mechanism 240 of the handle assembly 220 includes a hub member 242 and a plurality of flexible legs 244 that extend from the hub member 242 and are secured to the proximal portion 222b of the handle assembly 220. Although shown with ten (10) legs 244a, it is envisioned that the release mechanism 240 may have as few as two legs, or more than ten (10) legs. The plurality of flexible legs 244 may be formed of plastic, metal, or other suitable material. It is envisioned that the hub member 242 may be configured to facilitate introduction of the purse-string applicator 200 into the body cavity of the patient.

In an initial or unflexed condition (FIGS. 15 and 16), the plurality of flexible legs 244 form a bulbous shape with each leg 244a of the plurality of flexible legs 244 including an outwardly bowed portion. The plurality of flexible legs 244 are configured such that when the handle assembly 220 is fully received within the introducer sleeve 210, a proximal portion of the support ring 230 is received within an annular recess 217 (FIG. 16) positioned on a bulbous introducer portion 216 of the introducer sleeve 210, and the plurality of flexible legs 244 retain the support ring 230 against the bulbous introducer sleeve 210 within the recess 217.

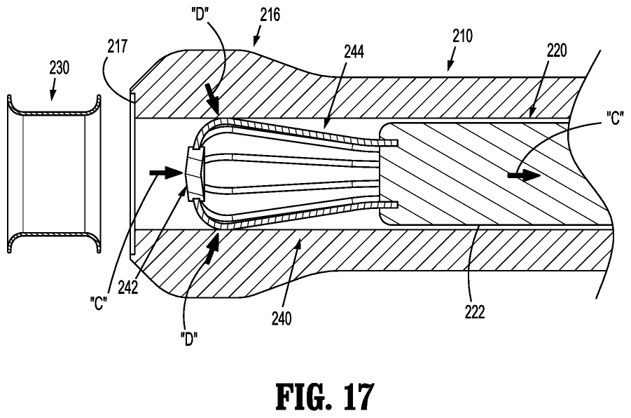

FIG. 17 illustrates the release of the support ring 230 from the purse-string applicator 200. More particularly, retraction of the handle assembly 220 relative to the introducer sleeve 210, as indicated by arrows “C”, causes a radially inward forces on the plurality of legs 244, as indicated by arrows “D” in FIG. 17, causing each leg 244a of the plurality of legs 244 to flex inwardly, thereby elongating the plurality of legs 244 and transitioning the plurality of legs 244 from the bulbous shape (FIG. 16) to a substantially cylindrical shape (FIG. 17). As the plurality of flexible legs 244 transition to the substantially cylindrical shape, the plurality of legs 244 move within the support ring 230 to release the support ring 230 from the purse-string applicator 200.

Persons skilled in the art will understand that the instruments and methods specifically described herein and illustrated in the accompanying drawings are non-limiting exemplary aspects of the disclosure. It is envisioned that the elements and features illustrated or described in connection with one exemplary aspect may be combined with the elements and features of another without departing from the scope of the disclosure. As well, one skilled in the art will appreciate further features and advantages of the disclosure based on the above-described aspects. Accordingly, the disclosure is not to be limited by what has been particularly shown and described, except as indicated by the appended claims.

What is claimed is:

1. A purse-string applicator comprising:
an introducer sleeve including a tubular body portion having proximal and distal portions, a handle portion disposed on the proximal portion, and an introducer portion disposed on the distal portion;
a handle assembly including an elongate shaft portion and a release mechanism, the release mechanism being positionable distal of the distal portion of the introducer sleeve; and
a support ring releasably engageable by the release mechanism of the handle assembly and including an annular body portion and first and second rim portions, the release mechanism releasably retaining the support ring adjacent the distal portion of the introducer sleeve.

2. The purse-string applicator of claim 1, wherein the introducer portion of the introducer sleeve includes a bulbous portion that is configured to facilitate entry of the bulbous portion into a body cavity of a patient.

3. The purse-string applicator of claim 2, wherein the introducer portion of the introducer sleeve defines a recess, the support ring received within the recess.

4. The purse-string applicator of claim 1, wherein the handle portion of the introducer sleeve is configured to facilitate grasping by a clinician.

5. The purse-string applicator of claim 1, wherein the elongate shaft portion of the handle assembly defines a pair of cutouts and the release mechanism includes a pair of ball-bearings received within the pair of cutouts.

6. The purse-string applicator of claim 5, wherein the release mechanism includes a pair of springs and the pair of ball-bearings are biased outwardly by the pair of springs into engagement with the support ring.

7. The purse-string applicator of claim 1, wherein the release mechanism includes a hub and a plurality of flexible legs extending from the hub.

8. The purse-string applicator of claim 7, wherein the plurality of flexible legs are transitionable between a bulbous condition and a substantially cylindrical condition.

9. The purse-string applicator of claim 8, wherein the plurality of flexible legs is configured to releasably engage the support ring when in the bulbous condition.

10. The purse-string applicator of claim 8, wherein retraction of the handle assembly relative to the introducer sleeve transitions the plurality of flexible legs from the bulbous condition to the substantially cylindrical condition.

11. A kit comprising:
an anvil assembly; and
a purse-string applicator comprising:
an introducer sleeve including a tubular body portion having proximal and distal portions, a handle portion disposed on the proximal portion, and an introducer portion disposed on the distal portion;
a handle assembly including an elongate shaft portion and a release mechanism, the release mechanism being positionable distal the distal portion of the introducer sleeve; and
a support ring releasable engageable by the release mechanism of the handle assembly and including an annular body portion and first and second rim portions.

12. A kit comprising:
a circular stapling instrument;
an anvil assembly releasably securable to the circular stapling instrument; and
the purse-string applicator of claim 1.

13. A method of performing an anastomosis using a double purse-string suture technique, the method comprising:
positioning a support ring into a tubular organ of a patient with a purse-string applicator;
securing a first section of the tubular organ to the support ring with a first purse-string suture;
separating the support ring from the purse-string applicator;
removing the purse-string applicator from the tubular organ;
positioning a distal portion of a circular stapling instrument, including a loading unit and trocar member, into the first section of the tubular organ such that the trocar member extends through the support ring;
positioning the support ring and attached first section of the tubular organ within the loading unit of the circular stapling instrument; and
actuating the circular stapling instrument.

14. The method of claim 13, wherein securing the first section of the tubular organ to the support ring includes forming the purse-string suture about an annular body portion of the support ring.

15. The method of claim 13, wherein separating the support ring from the purse-string applicator includes retracting the handle assembly of the purse-string applicator relative to an introducer sleeve of the purse-string applicator.

16. The method of claim 15, wherein removing the purse-string applicator from the tubular organ include retracting the introducer sleeve.

17. The method of claim 15, wherein positioning the distal portion of the circular stapling instrument into the first section of the tubular organ includes introducing proximal portion of the purse-string applicator through an anus of a patient.

18. The method of claim 15, further including securing the support ring to the purse-string applicator.

19. The method of claim 15, wherein actuating the circular stapling instrument includes stapling and cutting the tubular organ.

20. The method of claim 15, wherein actuating the circular stapling instrument includes forming an anastomosis donut.
